(19) United States
(12) Patent Application Publication
(10) Pub. No.: US 2014/0364847 A1
(43) Pub. Date: Dec. 11, 2014

(54) RESECTOSCOPE COMPRISING A SHAFT

(71) Applicant: OLYMPUS WINTER & IBIE GMBH, Hamburg (DE)

(72) Inventor: Simon Hirschfeld, Hamburg (DE)

(21) Appl. No.: 14/362,201

(22) PCT Filed: Oct. 30, 2012
(86) PCT No.: PCT/EP2012/004529

§ 371 Date: Jun. 2, 2014

(30) Foreign Application Priority Data
Dec. 15, 2011 (DE) 10 2011 121 159.8

(51) Int. Cl. A61B 18/14

(52) U.S. Cl. CPC A61B 18/149; A61B 2018/126
USPC 606/41

(57) ABSTRACT

The disclosure relates to a resectoscope comprising a shaft and an electrode arrangement that is mounted to be displaceable in a direction of the shaft, shaft, and a carriage that can be displaced in the same direction and that is provided with a carriage contact and with a receiving bore which extends parallel the shaft direction and into which the proximal end region of said electrode arrangement can be inserted by means of an electrode contact for contacting the carriage contact, contact, wherein one of the contacts is formed as a plug-in socket and the other one is formed as a matching plug-in pin, wherein the carriage contact is formed as a plug-in pin.

The invention relates to a resectoscope.

A generic resectoscope is illustrated in DE 10 2004 145 337 A1. Here, the carriage contact is formed as a plug-in socket and the electrode contact is formed as a plug-in pin. This construction corresponds to the previously pursued construction concept that emphasized on configuring the electrode arrangement as a disposable item as simple and inexpensive as possible and on arranging all structural elements that are more complex on the reusable carriage.

A plug-in pin is constructionally simpler and thus more cost-effective with respect to a plug-in socket which can be formed to be hollow and, for example, resiliently flexible by means of a slot.

Another generic construction is illustrated in DE 20 2005 013 853 U1. It can be seen here that by following the mentioned construction concept, in addition, a sealing O-ring is provided on the carriage and not on the electrode arrangement, as well as a latching device which likewise is arranged on the carriage.

However, this constructional concept pursued until now also leads to disadvantages. The carriage is a constructionally complex and expensive structural element which therefore usually is configured to be reusable. This means that after each use, said carriage has to be cleaned and sterilized. However, in the case of a plug-in socket arranged in a receiving bore of the carriage, cleaning is very difficult. Another disadvantage, due to the given very small dimensions, is the particularly thin-walled and fragile construction of the plug-in socket which easily can bend when inserting the plug-in pin, and consequently requires expensive repair of the carriage, for which the resectoscope has to be sent to a repair shop. Also, the contacts may overheat if, e.g., the contact resistance is increased due to contamination. In the case of the applied high-frequency currents to be transmitted, this can rapidly result in destruction. This too requires expensive repair works of the carriage.

It is an object of the present invention to avoid the mentioned cleaning and repairing problems.

According to the invention, the carriage contact is formed as a plug-in pin and, accordingly, the electrode contact is formed as a plug-in socket. Thus, the philosophy of the electrode arrangement being as simple as possible is dismissed, and after radical rethinking, a more complicated and expensive design of the electrode arrangement is allowed, which now requires the plug-in socket. This results in a more expensive electrode arrangement; however, it also results in significant improvements on the carriage. Now, the plug-in pin is located on the carriage. The plug-in pin is considerably easier to clean and, due to its simple construction, is less endangered by overheating and loss of contact material. Also, it is mechanically more robust and is less damaged when plugging in. The plug-in socket, which is more susceptible on all these points, is located according to the invention on the electrode arrangement. There, poor cleanability is not an issue since the electrode arrangement is a disposable item and does not have to be cleaned. Also, the mechanical and electrical susceptibility of the plug-in socket in the electrode arrangement is only a minor disturbance since the plug-in socket together with the electrode arrangement is replaced anyway after each use. The carriage formed according to the invention has a significantly reduced susceptibility to faults and a significantly improved cleanability. Overall, in terms of the electrode arrangement and also in terms of the carriage, this results in a reduction of susceptibility and therefore also in a reduction of repair and maintenance costs.

The invention can also be used for a bipolar electrode arrangement. For example, two plug-in sockets can be arranged in parallel on the electrode arrangement. However, the second electrode contact is advantageously arranged in a ring-shaped manner, which results in a more robust and a rotationally symmetric construction.

The second electrode arrangement requires resiliently flexible elements on one of the two sides to be contacted. Advantageously, radially resilient tongues are arranged for this purpose on the circumference of the electrode arrangement, wherein, again, the more complex and susceptible element is provided on the electrode arrangement.

Advantageously, an annular seal is also provided on the electrode arrangement and a resilient detent is likewise provided on the electrode arrangement.

The invention is exemplary and schematically illustrated in the drawing. In the figures:

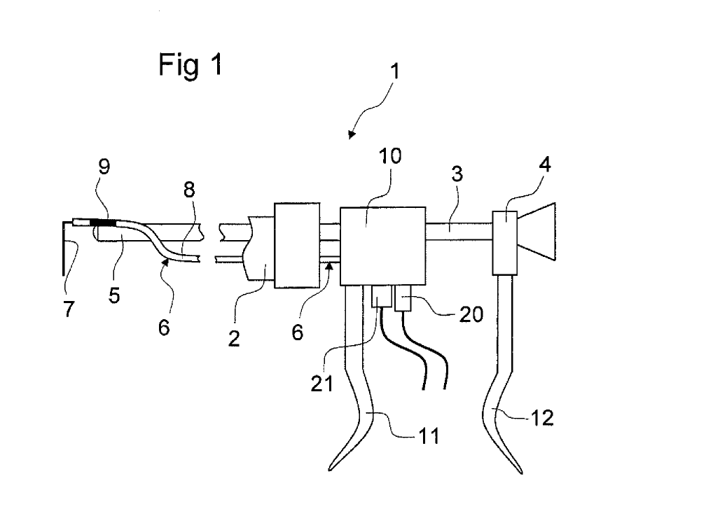

FIG. 1 shows the side view of a resectoscope according to the invention,

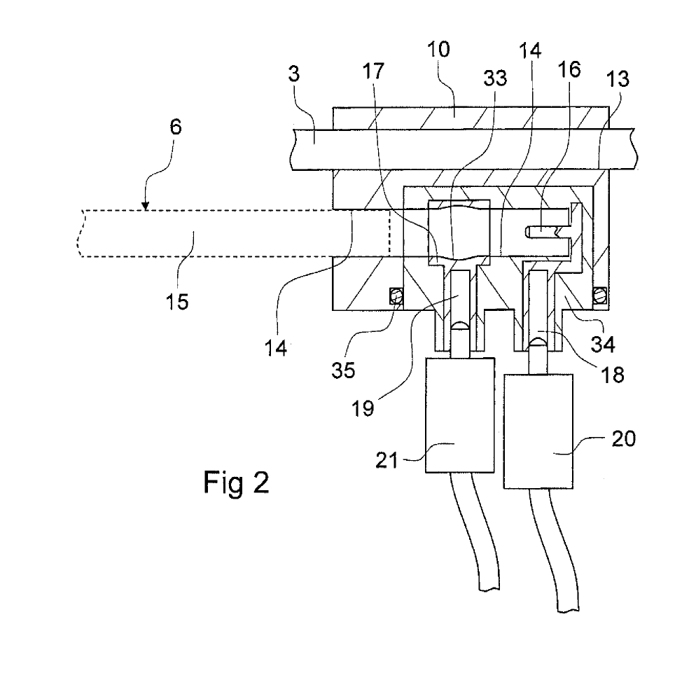

FIG. 2 shows in an enlarged longitudinal section the carriage of the construction of FIG. 1, and

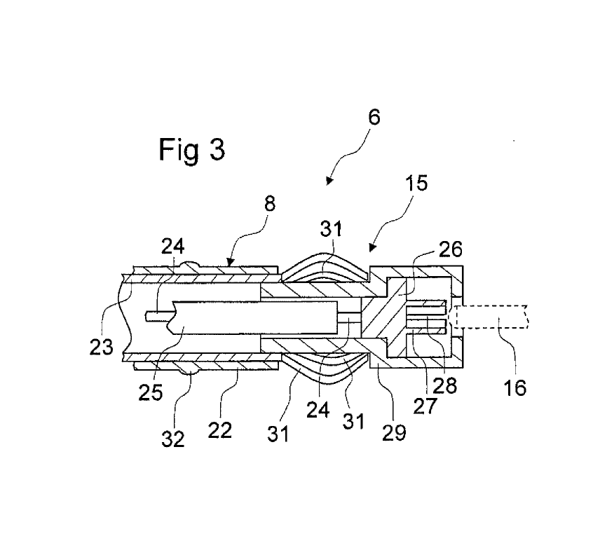

FIG. 3 shows in an enlarged longitudinal section the proximal end region of the electrode arrangement of the resectoscope of FIG. 1, wherein the thickness is more enlarged than the length.

FIG. 1 shows a side view of a resectoscope 1 comprising a shaft 2 that is connected at its proximal end via an optical guide tube 3 to a cover plate 4. Through this cover plate 4 and the optical guide tube 3, an optical system 5 can be inserted up into the shaft 2, from the distal end of which optical system the operation area to be operated with the resectoscope 1 can be viewed.

Furthermore, an electrode arrangement 6 is arranged in the shaft so as to be displaceable in the longitudinal direction of the shaft 2. At the distal end of the electrode arrangement 6, a cutting loop 7 is arranged in a configuration that is typical for resectoscopes. The cutting loop 7 is held by an electrode carrier 8 which, e.g., is formed in the usual fork shape in the distal end region. In the illustrated embodiment, an electric conductor 24 (FIG. 3) passes through the electrode carrier 8, which electric conductor is insulated with respect to the outside and forms the power connection for the cutting loop 7 that is to be acted on with high frequency.

In the distal end region of the electrode carrier 8, a return electrode 9 is formed as a region exposed to the outside on said electrode carrier. This return electrode is also contacted via a further conductor that passes through the electrode arrangement. The illustrated electrode arrangement 6 can also be configured in a monopolar configuration without the return electrode 9. Furthermore, the return electrode can also be arranged in a different form and at a different position. For example, the shaft 2 can form the return electrode.

The resectoscope 1 has a carriage 10 that is mounted to be displaceable in the direction of the shaft 2. In the usual construction that is used in the exemplary embodiment, the carriage 10 is mounted on the optical guide tube 3 in a longitudinally displaceable manner. With the handle pieces 11 and 12 on the carriage 10 and the cover plate 4, respectively, the carriage 10 can be moved in the proximal direction, wherein the cutting loop 7 performs the typical retracting movement by means of which the physician carries out the tissue-removing incisions, e.g., in the prostate, while the cutting loop is activated with high frequency.

FIG. 2 shows a longitudinal section of the inner construction of the carriage 10 which is guided by means of a guide bore 13 on the optical guide tube 3. Parallel to the guide bore 13 in the carriage 10, a receiving bore 14 is formed into which the proximal end region 15 of the electrode arrangement 6, illustrated in dashed lines, can be inserted.

At the proximal end of the receiving bore 14, a plug-in pin 16 protrudes in the axial direction into the receiving bore 14. Distally spaced apart from the plug-in pin 16, an annular contact 17 is arranged in the inner wall of the receiving bore 14. The plug-in pin 16 is positioned parallel to the axis of the receiving bore 14 and can be tapered in the distal direction so as to facilitate the insertion.

In the exemplary embodiment shown in FIG. 2, the plug-in pin 16 is electrically connected, namely integrally, to a socket 18. The annular contact 17 is electrically connected to another socket 19. As illustrated in FIG. 2, plug connectors 20 and 21 can be inserted into the sockets 18 and 19, which plug connectors connect via cables to a high-frequency generator. In this manner, the two poles of a high-frequency generator are connected to the electrodes 7 and 9 of the electrode arrangement 6.

For contacting the contact arrangement, illustrated in FIG. 2, in the carriage 10, the proximal end region 15 of the electrode arrangement 6 is formed as it is shown in FIG. 3 in a longitudinal section, in which the thickness is more enlarged than the length.

The electrode carrier 8, as already mentioned, is insulated on the outside, namely with an insulating cover 22. Said insulating cover encloses an electrically conductive inner tube 23 made of metal that forms the conductor which supplies the return electrode 9. At the location of the return electrode 9, the inner tube 23 itself can form the electrode by omitting the insulating cover 22 at this position.

The conductor 24 contacting the cutting loop 7 is arranged inside the inner tube 23 and is insulated with respect to said inner tube with an insulating sheath 25.

The proximal end of the conductor 24 is contacted with a contact piece 26, for example by soldering. The contact piece 26 is made of metal and carries a plug-in socket 27 that extends from the contact piece 26 in the proximal direction and has at least one slot 28 that extends in the axial direction and ensures that the plug-in socket 27 can resiliently flex in the radial direction. The plug-in socket 27 forms the proximal end of the electrode arrangement 6. Its inner diameter can be formed so as to increase in the proximal direction in order to facilitate the attaching process.

FIG. 3 shows in dashed lines the plug-in pin 16 still situated outside of the plug-in socket 27. By sliding the electrode arrangement 6 further in the proximal direction, the plug-in socket 27 is attached onto the plug-in pin 16 and contacts the latter in a clamping manner.

The contact piece 26 is arranged inside an insulating cap 29 which has a central hole through which the plug-in pin 16 can pass and which shields the contact piece 26 and the plug-in socket 27 from the outside in an insulating manner. The insulating cap 29 extends so far in the distal direction that it also encloses the exposed proximal end region of the conductor 24 in an insulating manner. In the exemplary embodiment, the insulating cap extends up into the proximal end region of the electrically conductive inner tube 23 and establishes a mechanical connection thereto as well as a continuous insulation.

At the proximal end of the electrically conductive inner tube 23, spring tongues 31 are arranged as a second electrode contact, which spring tongues, e.g., can be formed by longitudinally slotting the inner tube 23 in its proximal end region. The spring tongues 31 are curved outwardly and are resiliently flexible. When inserting the end region 15 of FIG. 3 into the receiving bore 14 of the carriage 10, illustrated in FIG. 2, up into the end position, the plug-in socket 27 thus is attached onto the pin 16, and the spring tongues 31 engage in a resiliently flexible manner on the inner surface of the annular contact 17. Accordingly, in the inserted position, the cutting loop 7 of the electrode arrangement 6 is connected to the plug-connector 20, and the return electrode 9 is connected to the plug-connector 21.

By the insulating cap 29, the insulating spacing is increased between the two proximal end contacts of the electrode carrier 6, namely between the plug-in socket 27 and the spring tongues 31, and a disturbing current flow between them is prevented.

In the exemplary embodiment, a sealing ring is provided which, when the end region 15 is inserted into the receiving bore 14, provides for sealing so that no liquid can penetrate inside the receiving bore 14. For this purpose, an elastic sealing ring is provided which radially outwardly protrudes the end region 15 and which, in the illustrated exemplary embodiment of FIG. 3, is formed as an annular bead 32 that is formed integrally with the insulating cover 22. The sealing ring can also be provided in a different formation and at a different position. For example, it can be mounted as an 0-ring in a circumferential groove that is formed at the location of the annular bead 32 in the insulating cover 22.

As FIG. 2 shows, the annular contact 17 in the illustrated exemplary embodiment has an inner surface that is slightly expanded in the middle with respect to the ends. This circumferential expansion forms a flat ring groove 33 into which the spring tongues 31 can snap in, thereby forming a resilient latching engagement that secures the end position of the insertion movement and ensures that the desired contact state is maintained. Reaching the correct latching engagement can be indicated to the user, e.g., through a latching sound or through a movement which, e.g., is clearly visible or can be sensed with the hand.

In the illustrated embodiment of the carriage 10, said carriage, as the aforementioned prior art shows, is provided with an interchangeable insert 34 by means of which the plug-in pin 16 and the annular contact 17 can be changed. An O-ring 35 that surrounds the insert 34 provides for sealing.

Instead of the cutting loop 7 shown in the figures, the electrode carrier 6 can also carry a differently shaped electrode, e.g., a flat vaporization electrode.

In order to convey an idea of the size of the above-described construction it is mentioned that the outer diameter of the proximal end region 15 of the electrode arrangement 6 preferably lies in a size range known from the prior art for such electrode arrangements of approximately 1.5 to 2 mm.

1. A resectoscope comprising a shaft and an electrode arrangement that is mounted to be displaceable in a direction of the shaft, and a carriage that can be displaced in the same direction and that is provided with a carriage contact and with a receiving bore which extends parallel the shaft direction and into which the proximal end region of said electrode arrangement can be inserted by means of an electrode contact for contacting the carriage contact, wherein one of the contacts is formed as a plug-in socket and the other one is formed as a matching plug-in pin, and the carriage contact is formed as a plug in pin

2. The resectoscope according to claim 1, comprising a bipolar electrode arrangement and two electrode contacts for contacting two carriage contacts, wherein the plug-in socket is arranged at the proximal end of the electrode arrangement and that a second electrode contact is arranged on the outside of the cylindrically formed end region of the electrode arrangement.

3. The resectoscope according to claim 2, wherein the second electrode contact has radially resiliently flexible spring tongues that are formed on the circumference of the electrode arrangement.

4. The resectoscope according to claim 1, wherein an annular seal for sealing the receiving bore is arranged on the end region of the electrode arrangement.

5. The resectoscope according to claim 1, wherein a resiliently flexible detent for latching into an indentation in the inner surface of the receiving bore is arranged on the end region of the electrode arrangement.

6. An electrode arrangement for use in a resectoscope according to claim 1, comprising an electrode at the distal end, and an electrode contact connected to said electrode at the proximal end, wherein the electrode contact is formed as a plug-in socket,

7. A carriage for use in a resectoscope according to claim 1, comprising a receiving bore into which an electrode arrangement can be inserted, and comprising a carriage contact that is arranged at the proximal end of the receiving bore and can be contacted by an electrode contact of the electrode arrangement, wherein the carriage contact is formed as a plug-in pin.
